(19) United States
(12) Patent Application Publication
(10) Pub. No.: US 2004/0138583 A1
(43) Pub. Date: Jul. 15, 2004

(54) Sincerity index system and program therefor

(76) Inventor: Anthony Michael Galea, Etobicoke (CA)

Correspondence Address: Siegmar Silber, Esq., SILBER & FRIDMAN, 66 Mount Prospect Ave., Clifton, NJ, 07012, US

(21) Appl. No.: 10/623,984

(22) Filed: Jul. 21, 2003

(30) Foreign Application Priority Data
Jul. 31, 2002 (CA) 2,396,202

(51) Int. Cl. A61B 5/05; A61B 5/103

(52) U.S. Cl. 600/547; 600/595

(57) ABSTRACT

A sincerity index system is provided for determining the sincerity of a person undergoing muscular testing. The system includes a muscular force detector for sensing and measuring the force exerted by first muscles upon a predetermined body movement and providing a first signal proportional to the force exerted. An electromyograph senses and measures the bioelectric activity of second muscles corresponding to the first muscles, the electromyograph providing a second signal proportional to the bioelectric activity. An interface device processes the signals and provides at least two factors, and an index-forming program combines the factors in a predetermined manner to provide a sincerity index having upper and lower limits. Using the system one can ascertain faked or exaggerated soft tissue injury, the extent of injury, and the extent of concomitant disabilities.

### BACKGROUND OF THE INVENTION

1. Field of the Invention

This invention relates to a system and program for measuring the sincerity/insincerity with which a person submits while undergoing muscular testing. Further, the system enables one to ascertain faked or exaggerated soft tissue injury and to determine the extent of injury and of concomitant disabilities. Additionally, with the increasing number of bogus insurance claims, the system provides an objective sincerity/insincerity index—the Galea index—derived from a physiologically based algorithm.

2. Background of the Prior Art

Measurement of muscular strength has been objectively assessed by the use of isokinetic principles. Such measurements have been used to follow rehabilitation training as well as to determine the extent or degree of temporary or permanent injuries of various muscles or muscle groups. During such muscular strength measurement or exercise, the muscles are loaded either statically or by applying dynamic forces. An isometric contraction occurs when a muscle force is exerted against a relatively immovable object. Then, the muscle under assessment does not move after the initial contraction. In the dynamic case, the muscle or muscles overcome an opposing force during the pathway of an entire muscular movement. Although such a force may for example be provided by weights or springs, devices permitting isokinetic contraction are particularly advantageous in this connection. An isokinetic contraction is one wherein a resisting force varying with the magnitude of the muscular force is applied and, simultaneously, a constant speed of muscular movement over the full range of movement is experienced. Thus, in measuring muscular force with such a device, as the muscular force is not uniform throughout the entire range of motion, the variation of the muscular force in the different parts of the range of movement is recorded at different speeds. Physiologically the muscle is strongest when fully extended, while, because of the anatomic levers, the body part controlled by the muscle is able to exert the mechanically greatest force in the mid range. In addition, when for example pain is experienced, the muscular force in a certain position may be impeded. Thus, as isokinetic measurement of muscular power gives considerably more information about the state of the muscles of a person than static measurement of muscular strength, the science and technology of isokinetics has seen considerable development, especially in the form of devices for applying and recording varying forces. Such development is summarized in standard sports physiology texts, which describe the expected normal distribution of forces applicable to muscle function assessment.

The accepted physiological explanation is that every coordinated movement requires the application of force by muscles that serve as the prime movers, the agonistic muscles, and a corresponding relaxation of antagonistic muscles that are, by nature, in opposition to the movement. For example, in elbow flexion, while the biceps muscle is agonistic and contracts with the application of force, the triceps muscle is antagonistic and relaxes during flexion.

The problem with isokinetic assessment of muscle function or dysfunction occurs when a patient being assessed does not put forth sincere effort. This may arise when, during an assessment of the muscles of the back, the patient who has been asked to move the apparatus forward, moves only slightly. The isokinetic unit cannot differentiate whether the reason for lacks of movement is because of dysfunction or because of lack of effort. The isokinetic unit alone can only partially answer this question.

Another aspect of this Background of the Prior Art is the present status of electromyographic measurement technology. In human beings, mechanical output (work) is achieved as a result of a willful, self-generated signal delivered to the sensory motor cortex from proprioceptive signals originating in muscles. In such instances the desire to produce work or motion is converted to an electrochemical signal which causes the contraction of certain appropriate muscle fibers and the relaxation of other appropriate muscles resulting in motion. The intensity of such muscle activity is transmitted by electrochemical means back to the brain or central nervous system where these intensity signals are compared to the signal. Any discrepancies are used to modulate or alter the contraction and relaxation of the muscles so as to bring the original stimulus and the resultant motion into conformity. This modulation is a continuous process which results in smooth motion. The transmission back is referred to as proprioceptive feedback and in the process, the intensity or presence of the muscle activity signals are compared to the stimulus. In humans afflicted with disease or physical trauma, the response to the feedback process changes and the change frequently prevents proper motion. Electrical activity measurement as a measure of muscle activity, electromyographic (EMG) measurements, which are used in a number of different medical applications, are performed using either an invasive or non-invasive technique. The use of invasive percutaneous EMG has widespread medical acceptance as an accurate technique for measuring electrical activity of an underlying muscle. However, invasive techniques require additional materials, expertise, and risk not found with non-invasive techniques. Measuring muscle activity using surface EMG (sEMG) measurements has attracted interest in scientists and medical practitioners for the last 30 years with its promise as an objective, non-invasive measurement (both percutaneous and non-invasive) in the treatment and assessment of dysfunctional muscle groups. An extensive body of scientific literature now exists describing the use of EMG measurements. However on its own clinical use of surface EMG has failed to produce a sufficiently objective evaluation of assessment of “sincerity of effort”. Static resting measurements are greatly influenced by small postural adjustment that cannot be adequately controlled. Accordingly, the postural and instrumental error frequently becomes so large so as to obscure useful information.

For the following initial discussion, the patents that are known to the inventor hereof in the fields of sincerity analysis, intentional muscle disability, and disability assessment are grouped together. In the Shah et al. patent, U.S. Pat. No. 5,184,628, a sincerity analysis is derived. Here, a grip analysis is performed wherein a load slope followed by a fatigue slope is compared to a predetermined force v. time characteristic. The deviation from this characteristic is adjudged to be indicative of sincerity. Shah et al. uses certain measurements of control groups and ratios of measured parameters form statistical lookup tables, the log transformations of which are then used for a sincerity index. The control group databases are drawn from either of healthy or diseased populations.

The patents to Brudny, U.S. Pat. No. 3,905,355 and to Cohen, U.S. Pat. No. 4,148,303, are now discussed. Cohen uses the term “intentional muscular disability” rather than “insincerity”. The Brudny patent, U.S. Pat. No. 3,905,355, is the forerunner of the Cohen development insofar as Brudny uses integrated EMG signals to develop vol appropriate willful muscular responses where impairment had affected volitional movements.

In U.S. Pat. No. 4,148,303 to Cohen, the neuromuscular activities of the muscles which are synergistic and antagonistic to the disabled muscle are measured while performing a specific movement or work task. The degree of activity is measured by recording the integrated EMG for each of the muscles. After these measurements are recorded, the corresponding neuromuscular activities of contralateral muscles are measured while performing the same movement or work task. Cohen determines the extent of purposeful disability by comparing the activation of the muscle which is synergistic to the disabled muscle with the degree of activation of the synergistic muscle which is contralateral thereto and by comparing the degree of activation of the antagonistic muscle with respect to its contralateral muscle while performing the work task and with the degree of activation of the muscle which is contralateral to the disabled muscle.

U.S. Pat. No. 4,890,495 to Slane teaches a device and a method for determining the push/pull capability of a subject to assist in the determination of “malingering” and to prevent the return of an individual to work too soon or in a position that may result in re-injury by providing objective and repeatable data on the actual performance ability of the subject in situations closely related to “real life” work conditions.

Additionally, the review of the foregoing patents, it is clearly seen that anthropometric statistics enter into evaluation of intent or sincerity analyses in most, if not all, of these patents.

In this section of the Background of the Prior Art the patents known to the inventor in the isokinetic dynamometer art is reviewed, including the descriptions of combined isokinetic dynamometry and chronophotography. U.S. Pat. No. 4,375,674 to Thornton provides a system for automatically gathering three-dimensional, kinesimetric data on human and machine subjects. The system combines data from a CYBEX II Isokinetic Dynamometer (Lumex, Inc., Bay Shore, N.Y.) and an array of RCA Model TC-1000 video cameras. The nature and format of the data acquired are such that the data may be utilized automatically and directly to study performance and environment design. The patent teaches utilizing the data to determine complete motion parameters of subject bodies and body portions in terms of position, velocity, acceleration and/or force, or any combination thereof. Individual and characteristic data sets are derived describing bodily motions, and presented in the form of envelopes of reach, functional reach, velocity, acceleration, and force, for example. Particular motions can also be tracked or recorded in terms of any or all of the aforementioned parameters. Displays, diagrams or other data are generated of any selected region or aspect of the aforementioned data sets, including a planes, volumes, regions or trajectories, and which are either manually or automatically derived. The data is manually or automatically interfaced with external physical environmental information, and graphic displays or other aspects of the interaction automatically or manually generated. Here combining photographic and isokinetic records is used in design evaluations.

U.S. Pat. No. 4,462,252 to Smidt et al. teaches the adaptation of the CYBEX II equipment for use as a trunk dynamometer. The device has two modes of operation, namely isometric (static) and concentric (dynamic). In the isometric mode of operation, the trunk position is controlled and the moment applied to the dynamometer by the flexors and extensors of the spine is measured. In the concentric mode of operation, the angular velocity and range of motion of the spine are controlled while the moment applied to the dynamometer by the active shortening of the flexors and extensors of the spine is measured. In both cases, analog recordings of the moment vs. time are made in real time. Concentric exercise is initiated with the trunk pad frame resting against the extension displacement stop. Subjects are instructed to perform concentric exercise and until the investigator terminates the test. By observation of the first six (6) cycles, estimates of the mean peak flexion and extension moments are made. These values are halved and the results are recorded. The recorded values represent an estimate of a 50% (fifty per cent) decrement in mean peak moment. The subject continues concentric exercise until peak moment values for both flexors and extensors decrease to the estimated 50% decrement. At that limit, the subjects are instructed to relax. From the data gathered, muscular strength and endurance is evaluated.

U.S. Pat. No. 4,885,939 to Martin describes a CYBEX human performance dynamometer. The dynamometer allows for three primary modes of testing: a first mode providing for concentric muscular contractions in both directions of rotation of an input shaft of the dynamometer; a second mode providing for concentric contractions in one rotational direction of the input shaft and eccentric contractions in the other; and a third mode providing for eccentric contractions in both directions of rotation of the input shaft.

The next group of patents known to the inventor refer to electromyographic (EMG) measurement as introduced above. The patents, while generally providing a muscle function analysis system, disclose various applications, namely, but not limited to, providing feedback for teaching conscious control over muscle activity; correcting spastic, paretic, or other muscle impairment; determining the effectiveness of muscle therapy; tracking the level of worker fatigue; detecting injuries in the lumbar spine of a patient; and monitoring back muscles. The earlier work in this area arises from the Stulen and DeLuca patent, U.S. Pat. No. 4,213,467, which, in turn, references DeLuca's A Polar Technique for Displaying EMG Signals, 28 ACEMB, New Orleans, 1975. More recently, De Luca et al. have advanced the art through U.S. Pat. Nos. 5,085,225; 5,085,226; 5,086,779 and 5,163,440.

U.S. Pat. No. 3,641,993 to Gaarder et al. discloses an electrical apparatus which measures human muscle activity and provides both visual and audio information relative to such activity for feedback to the human. Slight skin voltages caused by muscle activity are assumed by electrodes, transmitted via shielded electrical leads and processed by the apparatus, first to provide a visual display of the instantaneous logarithm of the peak value of such signals and secondly to provide a visual display of an accumulation or integration of the instantaneous logarithm of the peak signal values over a selectable and timed integration interval.

In U.S. Pat. No. 4,664,130 to Gracovetsky, a noninvasive method for the detection of a mechanical abnormality or injury in the lumbar spine of a patient is disclosed. The method combines both visual and electromyographic techniques. Also described is equipment for the detection of mechanical injuries in the lumbar spine of a patient and for the identification of the injuries as the compression and/or torsional type. The equipment derives a representation of the anatomy of the spine that includes as possible support for any moment of force generated onto the spine by the body weight and any external load carried by the patient, the posterior ligamentous system. Further, this representation of the anatomy of the spine also includes the extensors of the hip, which have the power and the leverage to flex the spine.

U.S. Pat. No. 4,667,513 to Konno discloses an electromyograph for detecting muscle fatigue. In this device, a monitor displays the dorsal muscular strength, graph, processed from EMG measurements, and, at the same time, the EMG signals produced by the muscle under testing. From the data detection of fatigue and pain of the muscles can be detected.

A continuing problem for insurance companies has been the number of individuals who claim more severe injuries from an accident than what have in fact occurred. In many cases it is quite difficult to determine whether or not the individual is sincere in his claim. A system and program has now been developed to assist insurance companies, attorneys and physicians in determining how such claims should be handled.

### SUMMARY OF THE INVENTION

In general terms, the invention disclosed hereby includes a sincerity analysis system for determining the sincerity of a person undergoing muscular testing. The individual is asked to take some tests involving moderate physical activity. The tests are monitored by instruments, and the results of the tests are analyzed by a computer program called the Galea Sincerity Analysis program. It is important to note that the program does not require comparison to normative data banks with statistically gathered anthropometric. The analysis is based on physiological accepted principles applied to individual being tested. The analysis employs a muscular force detector, such as an isokinetic dynamometer, for sensing and measuring the force exerted by a specific set of muscles during a predetermined body movement and also employs an electromyograph for sensing and measuring the bioelectric (EMG) activity of a second set of muscles that are antagonistic to the first set of muscles. The two measuring devices—the electromyograph and the isokinetic dynamometer—are operated simultaneously during the predetermined body movement. The signal outputs proportional to the measured force exerted and the measured EMG are further processed by a comparator which analyzes the signals and derives a correlative value or index between 0 and 1 reflecting the sincerity of effort. The system further includes software, which with the information from both the isokinetic unit and the sEMG unit, analyzes the data and produces a calculates the above-mentioned index during the testing protocol reflecting insincere, submaximal or maximal effort.

### OBJECTS AND FEATURES OF THE INVENTION

It is an object of the present invention to provide a sincerity analysis of a person undergoing muscular testing, which analysis correlates with physiological data.

It is a further object of the present invention to provide a sincerity index that is determinable from muscular force and electomyographic data.

It is yet another object of the present invention to provide a computer program to calculate the sincerity index while testing is in process.

It is still yet another object of the present invention to provide a system that simultaneously measures the muscular force exerted by one set of muscles and the bioelectric output from a second set of muscles contralateral to the first set.

It is a feature of the present invention that the system is applicable to injuries various parts of the body, including the lower back, knee, wrist, elbow and shoulder.

It is another feature of the present invention to have a test protocol that is useful in disability claims and for rehabilitation work.

It is yet another feature of the present invention to have a sincerity test which is reproducible.

Other objects and features of the invention will become apparent upon review of the drawings and the detailed description which follow.

### BRIEF DESCRIPTION OF THE DRAWINGS

In the following drawings, the same parts in the various views are afforded the same reference designators.

FIG. 1 is a system block diagram for the sincerity analysis system of this invention showing the comparator unit for processing simultaneously signals from a muscular force dynamometer and an electromyograph;

FIG. 2 is a flow chart showing the principal functional elements of the sincerity analysis system of the invention shown in FIG. 1;

FIG. 3 is a flow chart showing details of the CALCULATE TIMING FACTOR function of FIG. 2;

FIG. 4 is a flow chart showing details of the GETINACTIVE RATIO function of FIG. 3;

FIG. 5 is a flow chart showing details of the CALCULATE FATIGUE FACTOR function of FIG. 2;

FIG. 6 is a flow chart showing details of the FATIGUE CHECK CHANNEL function of FIG. 5;

FIG. 7 is a flow chart showing details of the CALCULATE FORCE SINCERITY function of FIG. 2;

FIG. 8 is a flow chart showing details of the CHECK ASD ROWS function of FIG. 2;

FIG. 9 is a flow chart showing details of the CHECK TORQUE vs. SPEED function of FIG. 2;

FIG. 10 is a flow chart showing details of the CHECK POWER vs. SPEED function of FIG. 2;

FIG. 11 is a flow chart showing details of the CHECK ECCENTRIC vs. CONCENTRIC function of FIG. 2; and

FIG. 12 is a schematic view of a patient connected to the isokinetic dynamometer and to the surface electromyograph of FIG. 1.

### DESCRIPTION OF THE PREFERRED EMBODIMENTS

The sincerity index system and program therefor of this invention disclosed herein pertains to the simultaneous measurement of muscular forces involved in a specific movement or task. In every movement one set of muscles operates to balance that of a second set of muscles—one set pushes, the other pulls. In the course of the description hereinbelow, an arrangement of measuring simultaneously one set of agonistic muscles with a dynamometer and the corresponding antagonistic set of muscles with an electromyograph is presented in detail. The dynamometer is a muscular force detector which senses and measures the force exerted by a muscle or set of muscles upon a predetermined body movement. The force detector has a signal output proportional to the force exerted. The electromyograph senses and measures the bioelectric (EMG) activity of the corresponding second muscle or set of muscles. The electromyograph, simultaneously with the force detector, provides a second signal output proportional to the EMG. The outputs from the two instruments are then processed at an interface or comparator unit and at least two factors for correlating the values derived from the processed signals are provided. The Galea sincerity index is then formulated by a program, described in detail below, which program combines the factors in a predetermined, weighted manner to provide a sincerity index having a lower limit of 0 and an upper limit of 1.

In the system and program therefor that follows, a presently developed isokinetic dynamometer and a presently developed surface electromyograph are used as system components. These units have computers integrated therewith and, in turn, a third computer is used to provide the sincerity analysis as presented herein. It is anticipated that with further development of this system the functions requiring the aid of a computer will be merged eventually into a single processing unit. Although there are numerous dynamometers for human muscle testing, the protocol herein uses a CYBEX Model 6000 Extremity System (manufactured by CYBEX, a Division of Lumex, Inc., Ronkonkoma, N.Y. 11779) isokinetic dynamometer. This dynamometer measures both eccentric contractions and concentric contractions and is well-described in the patent literature. Reference is made to the patent to M. Martin, U.S. Pat. No. 4,885,939 which describes the modular components added to the basic CYBEX unit to adapt the same for both eccentric and concentric contractions. This isokinetic dynamometer is equipped with modular components for testing the musculature of various parts of the body. Similarly, as to the electromyograph a unit manufactured by NORAXON USA, Inc. Scottsdale, Ariz. 85254 is used either in the fixed-cable arrangement, Model 1200, or in the telemetry arrangement, Model Telemyo. The signal processing of the Noraxon units is described in U.S. Pat. No. 4,999,584.

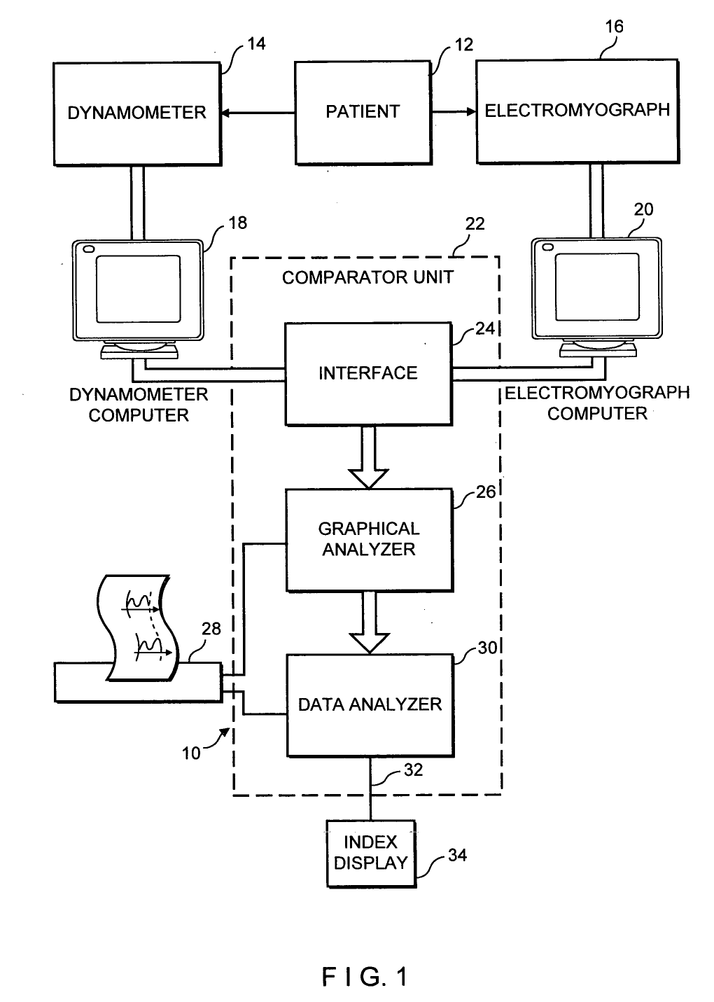

Referring now to FIG. 1, a system block diagram for the sincerity analysis system 10 is shown. The person 12 undergoing muscular testing is connected to an isokinetic dynamometer 14 by which the muscular force of one set of muscles associated with a body movement is monitored. The muscles antagonistic thereto are, in turn, connected to an electromyograph 16.

Each instrument records results with the aid of the associated computers 18 and 20, respectively. Once recorded, these results are retrievable in both numeric and graphical form. The results are maintainable on the hard disks of the computers or on associated memory devices.

The dynamometer and the electromyograph are operated simultaneously and are in synchronicity as the measurements of different aspects of the same physical actions are collected. Furthermore, any synchronization adjustment of the outputs of the computers 18 and 20, which are connected to the comparator unit 22, is accomplished by the interface 24 thereof. At the graphical analyzer 26, several graphs of the readings from the dynamometer 14 and the electromyograph 16 are plotted in a vertical array, that is one above the other, such that a vertical line on the printout connects several simultaneous measurements. This graphic display allows visual correlations of various factors. This collection of correlative data also provides data for the calculation of the degree of sincerity applicable to the physical action being tested. In the sincerity analysis system 10, the data analyzer 30 processes the signals from dynamometer 14 and the electromyograph 16 and calculates, using an algorithm, an index that takes into account various factors delineated hereinbelow. The output 32 of the data analyzer 30 is the sincerity index or Galea index and is displayed at index display 34. The index is a value in the range from zero to one. A value of one indicates that the individual being tested is sincerely performing the physical actions to the best of his/her ability. A value of zero indicates that no effort to perform the physical actions is being made. That is to say that one signifies total sincerity, and zero indicates total insincerity. Values between zero and one indicate proportional degrees of insincerity/sincerity.

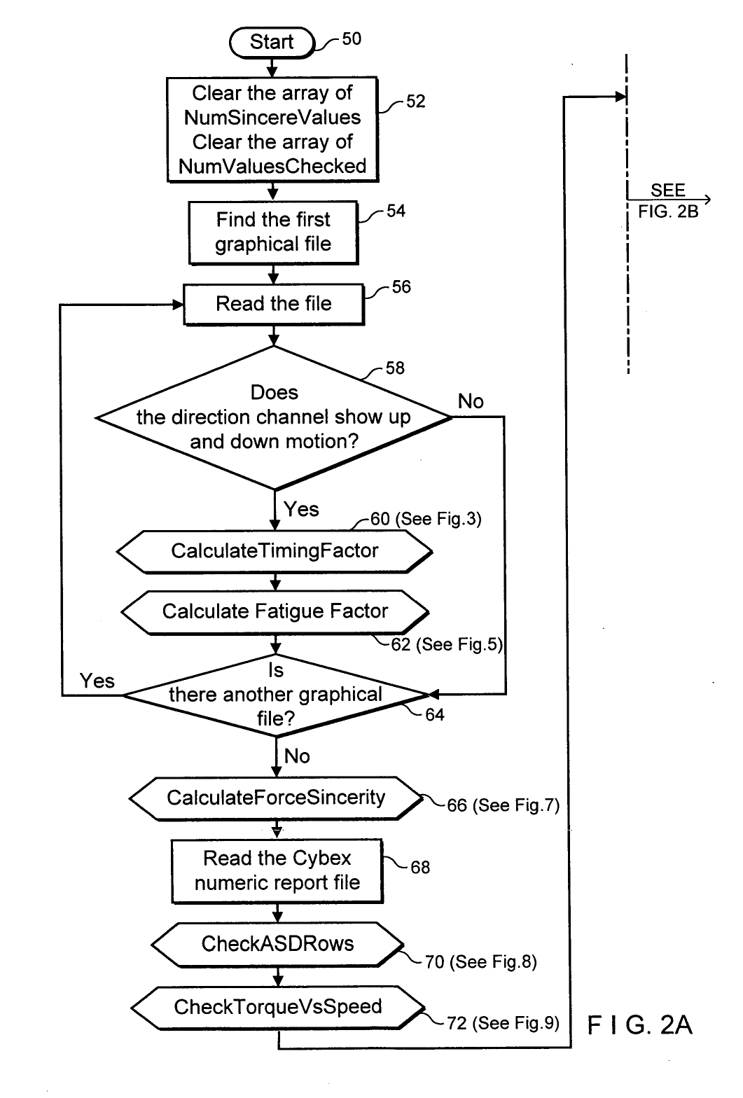

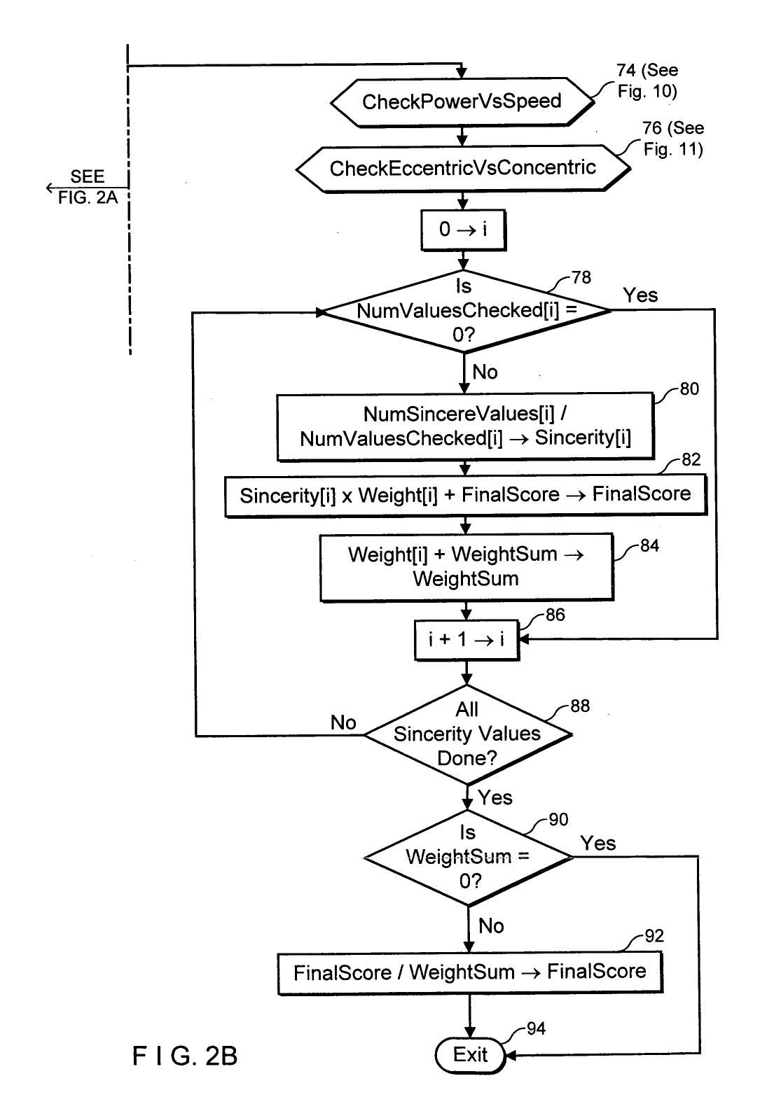

Referring now to FIG. 2 a flow chart is shown providing the details of the principal functional elements of the sincerity analysis system of this invention. The flow chart for the sincerity analysis program 40 is provided with the test result data from the computers of the dynamometer 18 and the electromyograph 20. While the electromyograph computer 20 generates primarily a graphical database, the isokinetic dynamometer computer 16 performs extensive computations in its own right, and creates a database containing the digital results of these calculations. These two databases are analyzed by the sincerity analysis program. As a separate graphical file is created for each separate physical test that is performed upon the individual, numerous files become available for analysis by the sincerity analysis program.

To aid in understanding the system, it is indicated that the sincerity index or the Galea index is the result of combining several sincerity factors. In the example at hand, seven factors are analyzed and each sincerity factor is a value, which varies from zero to one. Although all seven factors are used, any combination in which at least one factor is derived from the dynamometer and another from the electromyograph may be used to derive the index. Each sincerity factor is multiplied by a weighting value, and these products are summed to give the final result. Each weighting factor is also a value between zero and one, and the sum of the weighting factors is one. Thus the final result also varies from zero to one. Although the index calculation is here arbitrarily weighted to provide a result which varies from zero to one, other weightings for an index that vary, for example, between zero and ten or zero and one hundred are within the scope of this invention. In general terms, the sincerity index is provided by the formula:

S=ΣF W and W=0≦F≦1, 0≦W≦1, 0≦S

where S is the final result,

the F are the sincerity factors, and

the W are the weights.

Using the above factors, the algorithm for the Galea index is manually computable. However, using the data analyzer 30, the program thereof generates the index during testing which, in turn, gives the test provider greater flexibility. The computer-generated index is next described.

The sincerity analysis program shown hereinbelow calculates seven factors which are then combined into a final sincerity score. Each factor is calculated by testing several values. Each test makes a decision as to whether the corresponding value indicates sincerity or not. The value of the factor is the ratio of the number of tests which came out sincere divided by the total number of values tested. If all the values came out sincere then the factor has a value of one. If none of them tested sincere then the factor will have a value of zero.

The main flow of the procedure is indicated on the flow chart, FIG. 2, and starts at START block 50. First, the two arrays NUM SINCERE VALUES and NUM VALUES CHECKED block 52 are cleared to zero. Each of these arrays has an entry for each factor. NUM SINCERE VALUES contains the numerators of the factors, and NUM VALUES CHECKED contains the denominators of the factors.

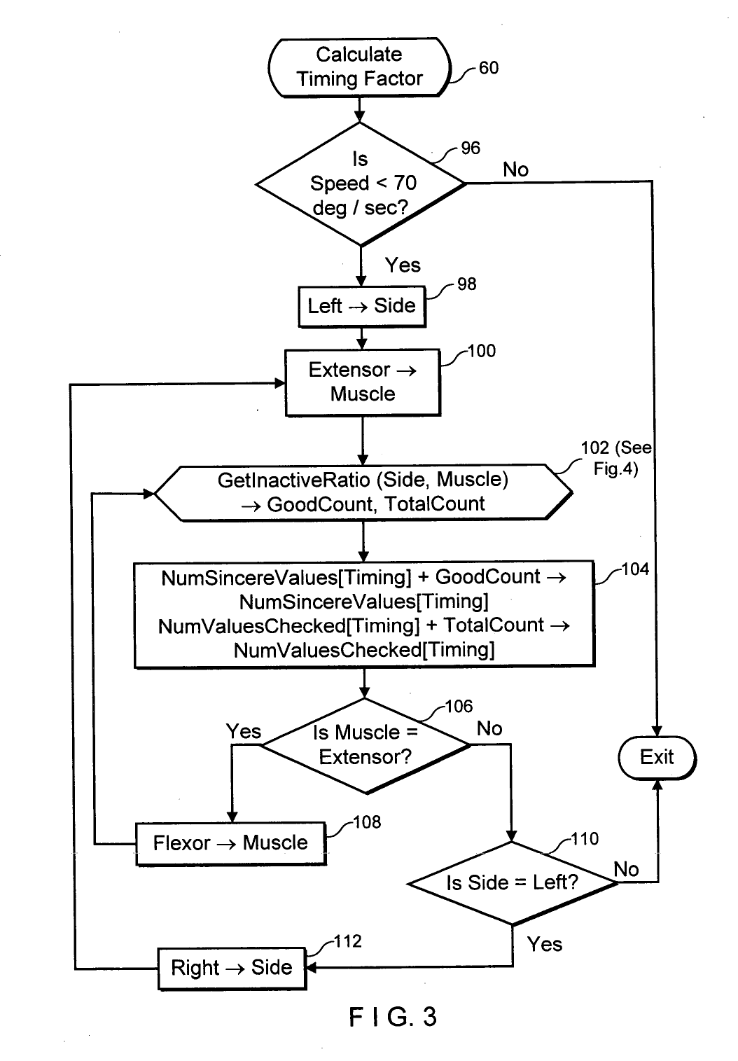

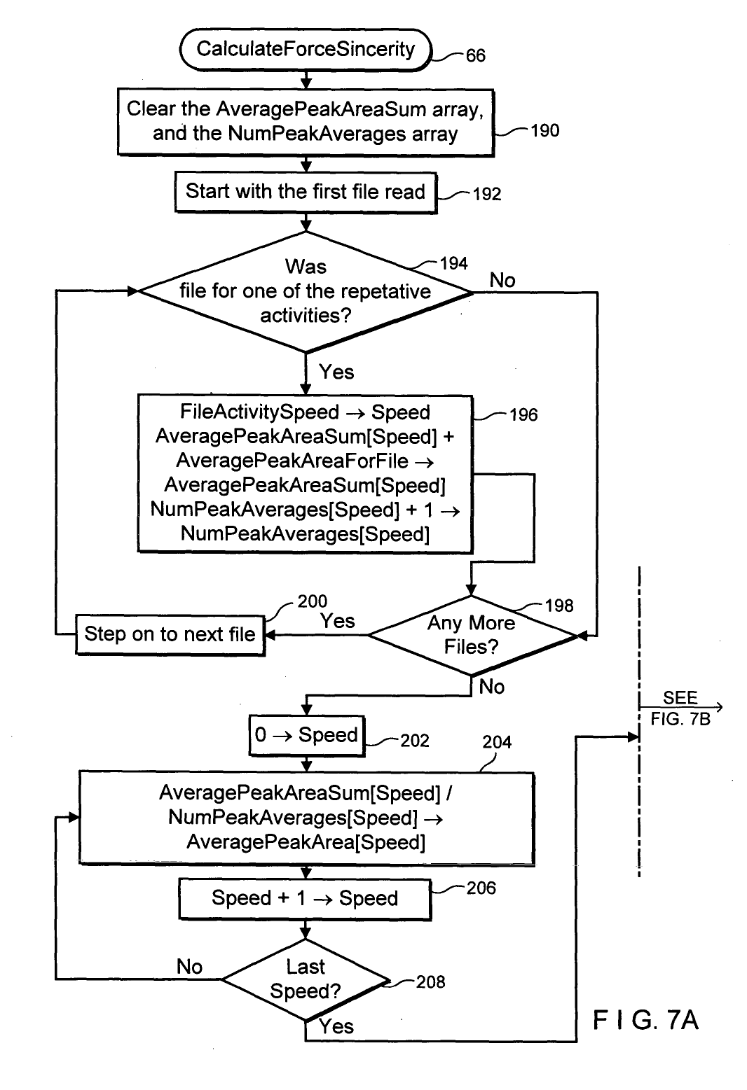

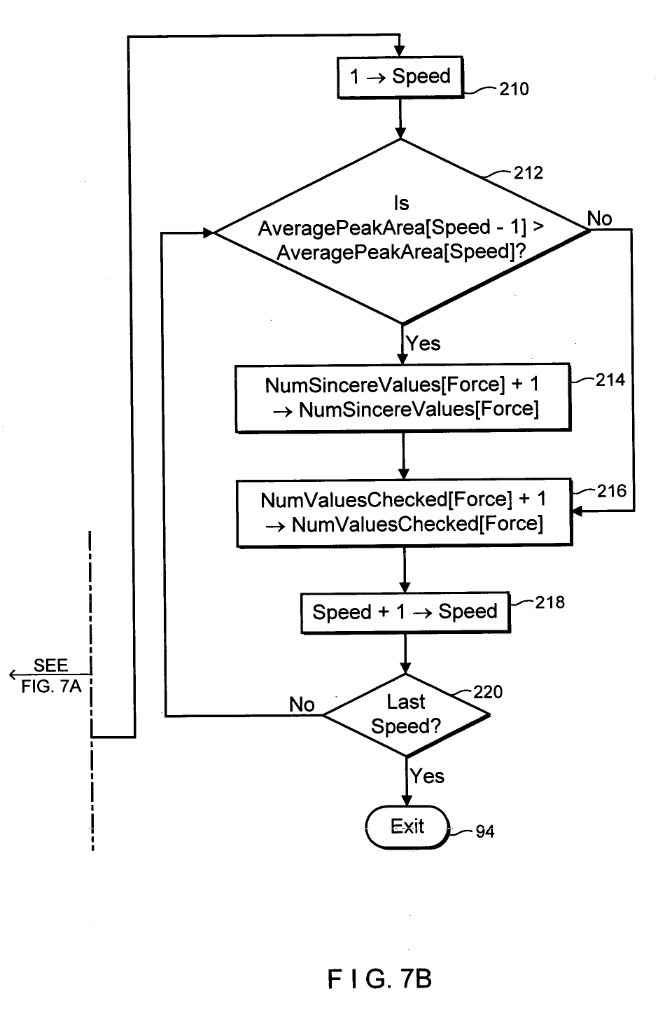

Next, at FIND THE FIRST GRAPHICAL FILE block 54 a search is made for the graphical files that are created while the subject is being tested. Each of these files is read at READ THE FILE block 56 by the program one by one. The information required to calculate three of the factors is extracted from the files as they are read. Then at the DOES THE DIRECTION CHANNEL SHOW UP AND DOWN MOTION? block 58, each file is examined to see if it contains a direction channel which shows a repetitive, up and down motion. If not, the file is discarded and a search is made for another file. If up and down motion is indicated, then the file is used immediately to calculate the contribution made by that file to the timing factor, and the fatigue factor. This is done by the functions CALCULATE TIMING FACTOR and CALCULATE FATIGUE FACTOR blocks 60 and 62, respectively. These factors are described in detail in FIGS. 3 through 6 hereinbelow. Information from the file is saved enabling the later calculation of the force factor. Once all the graphical files have been read and analyzed, the numerator and denominator of the force factor are calculated by the function CALCULATE FORCE SINCERITY block 66. (See FIG. 7 hereinbelow.).

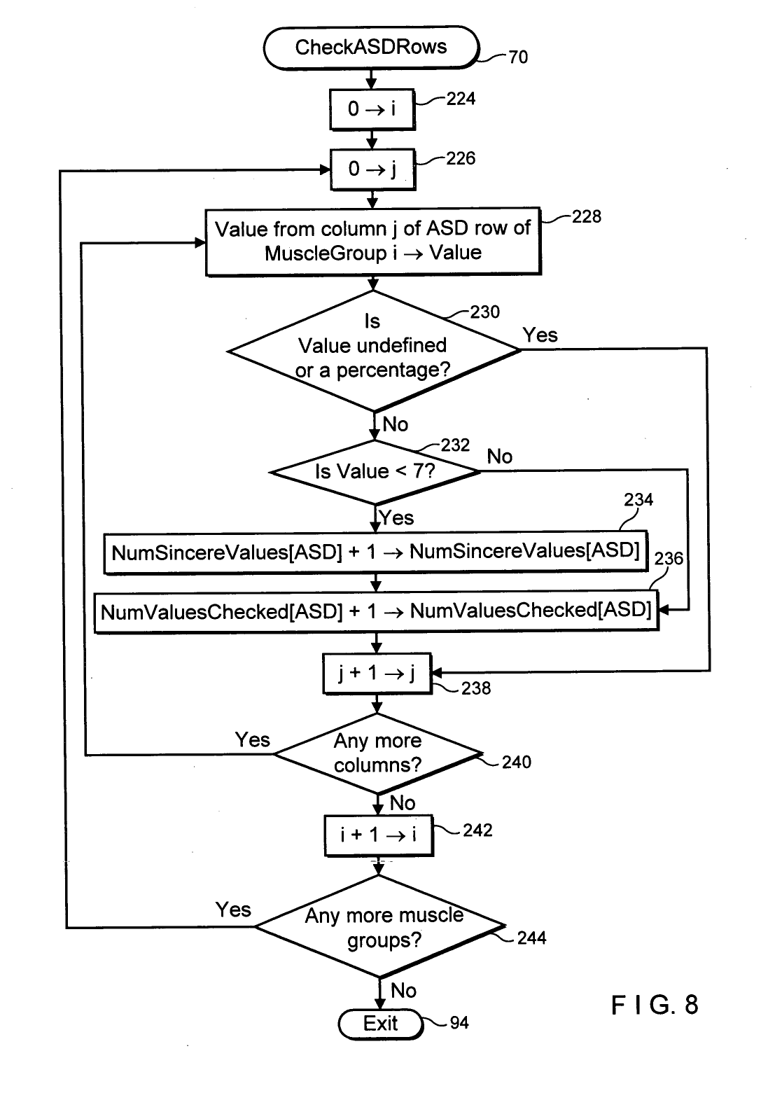

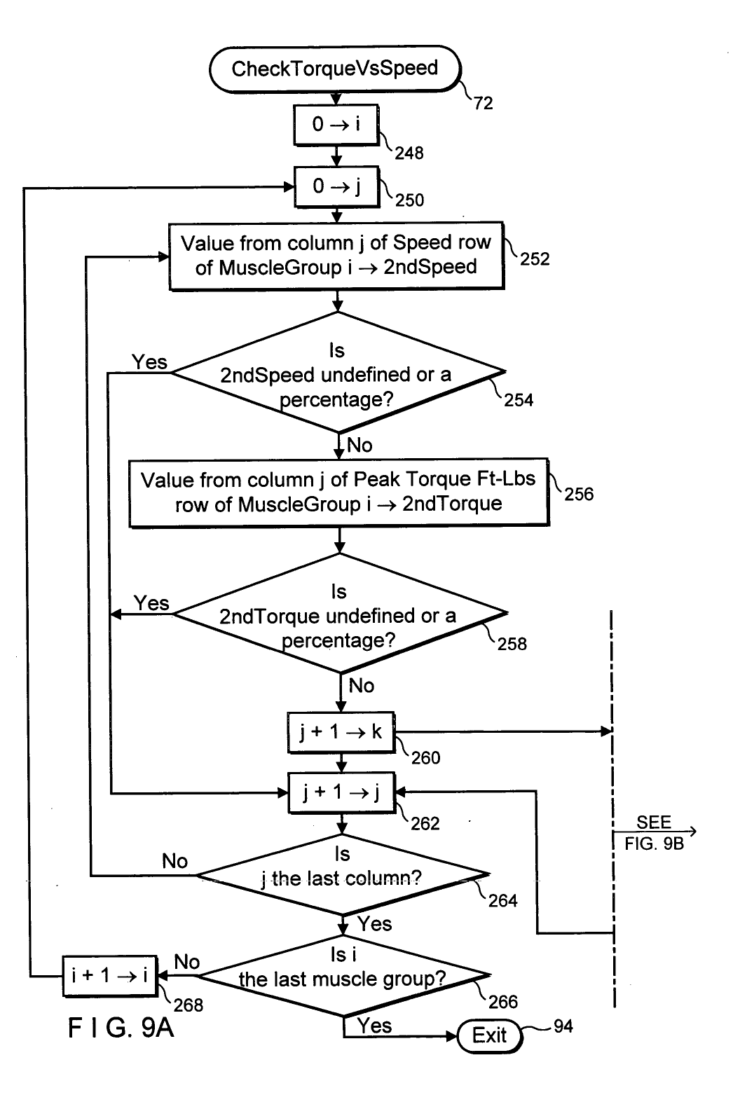

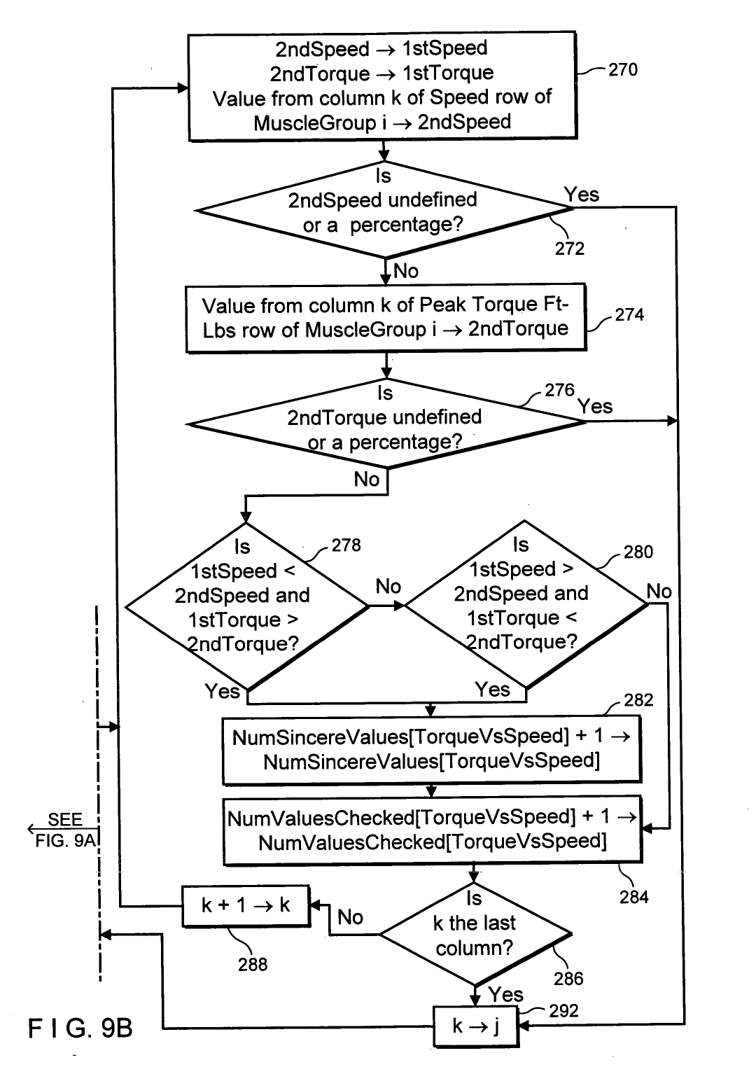

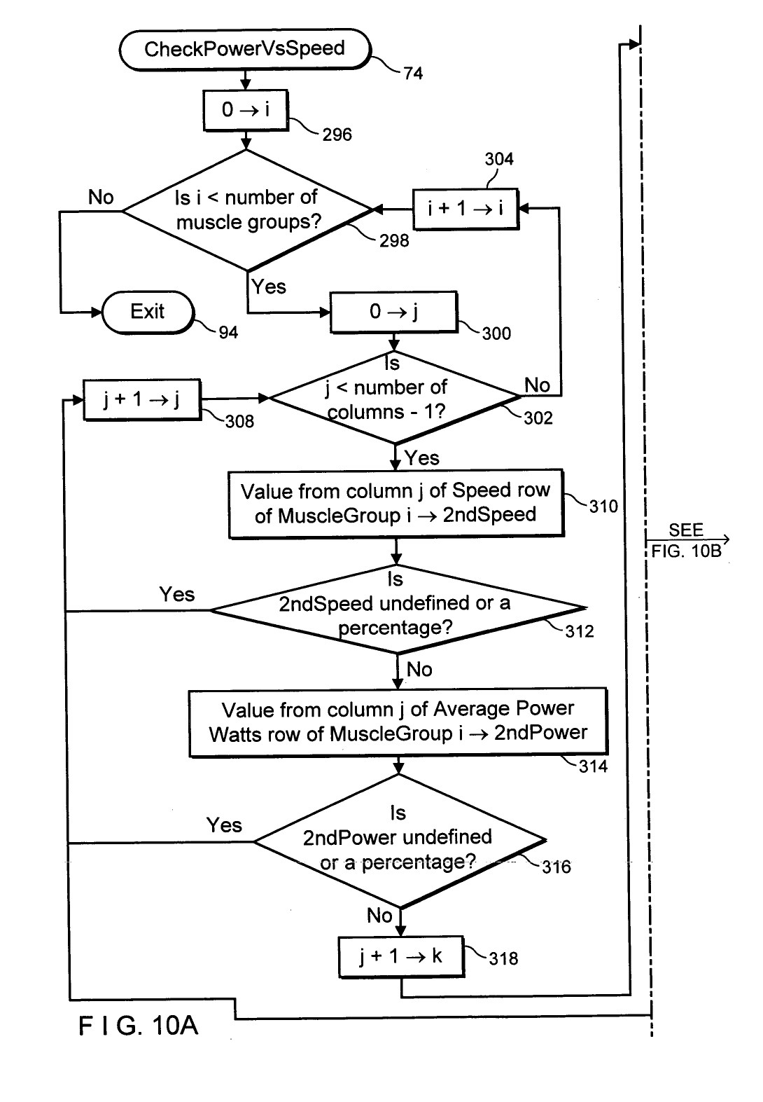

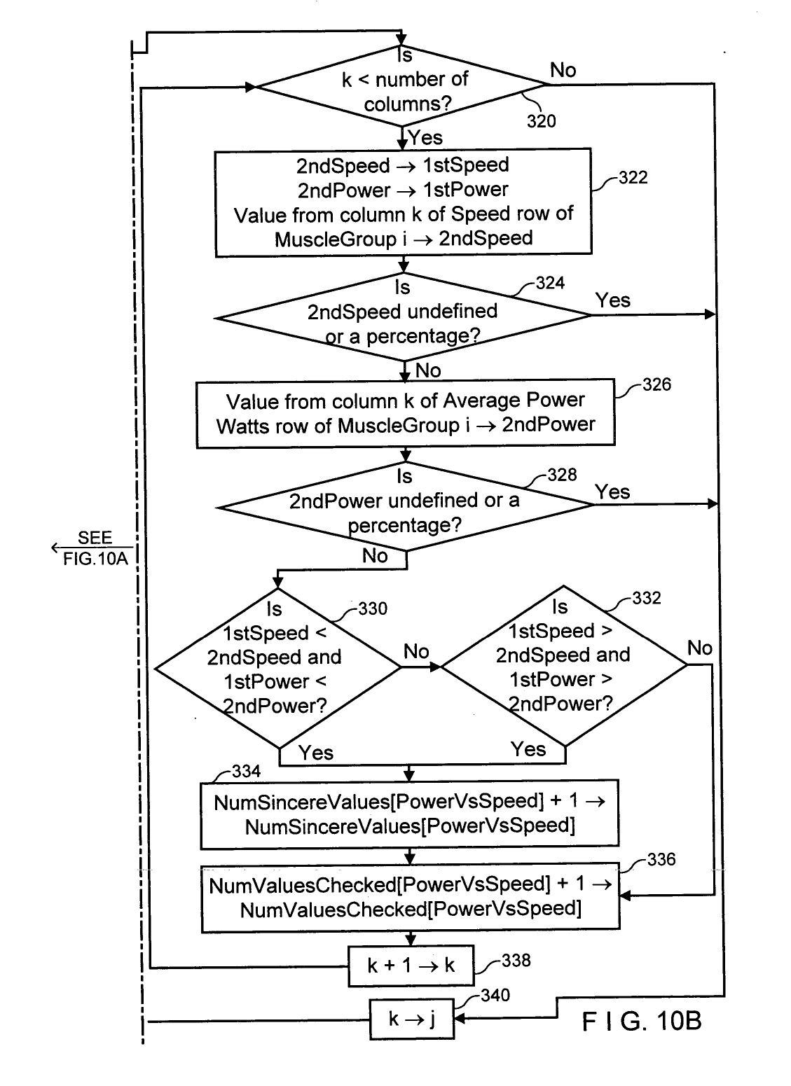

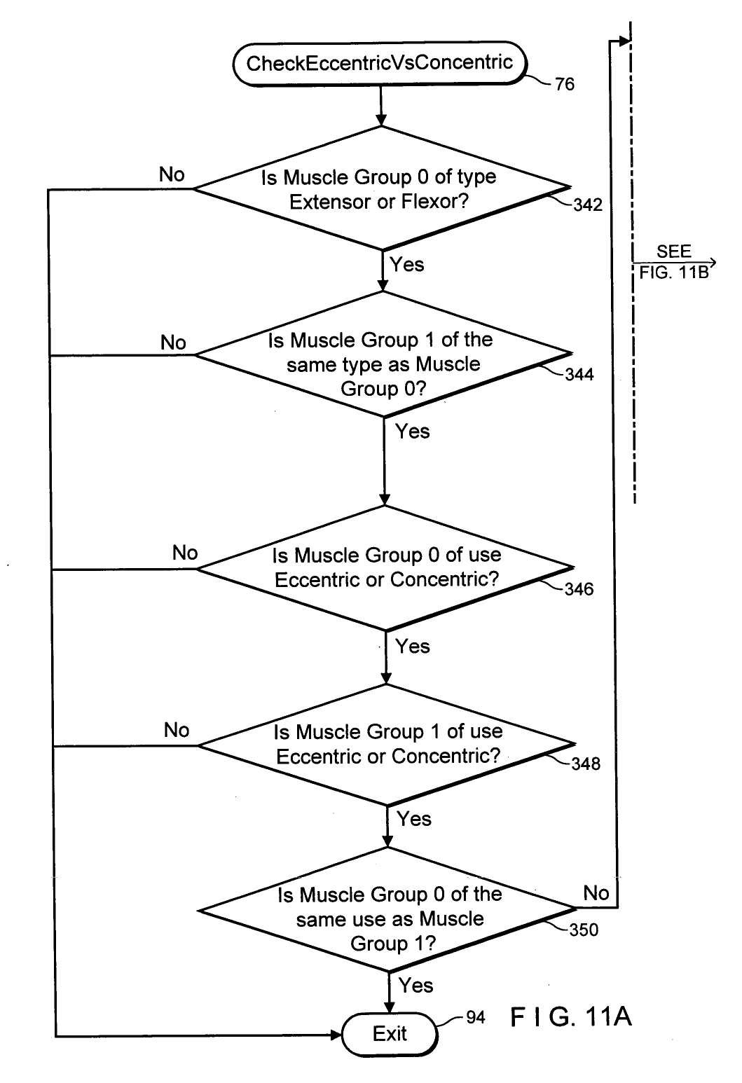

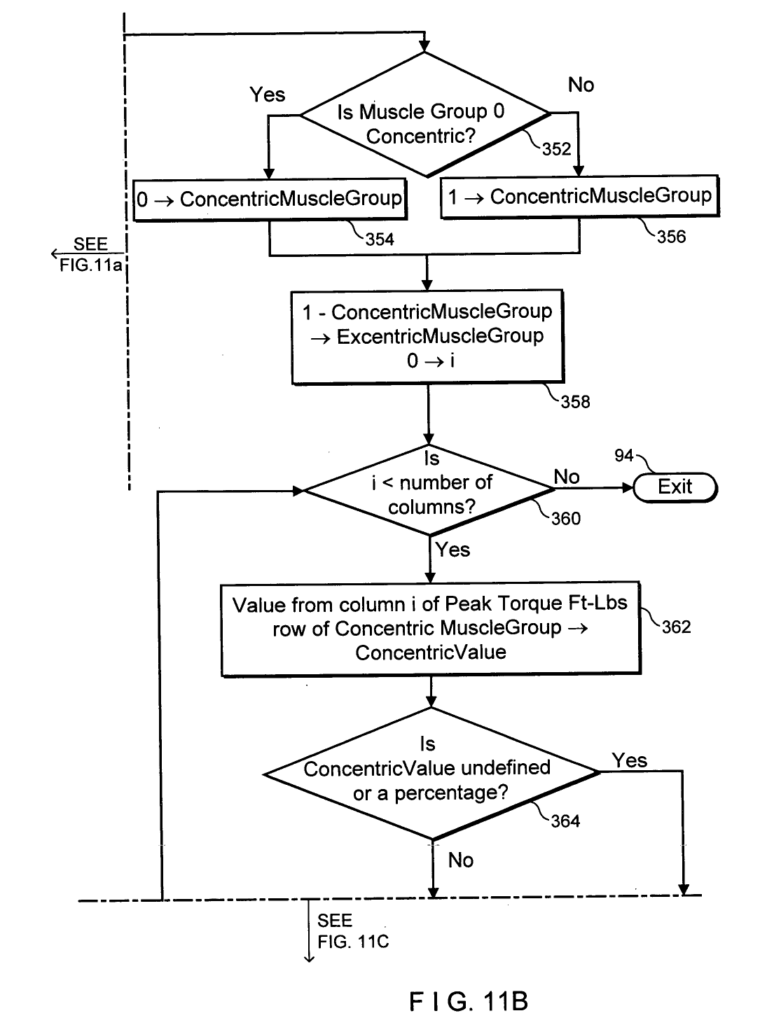

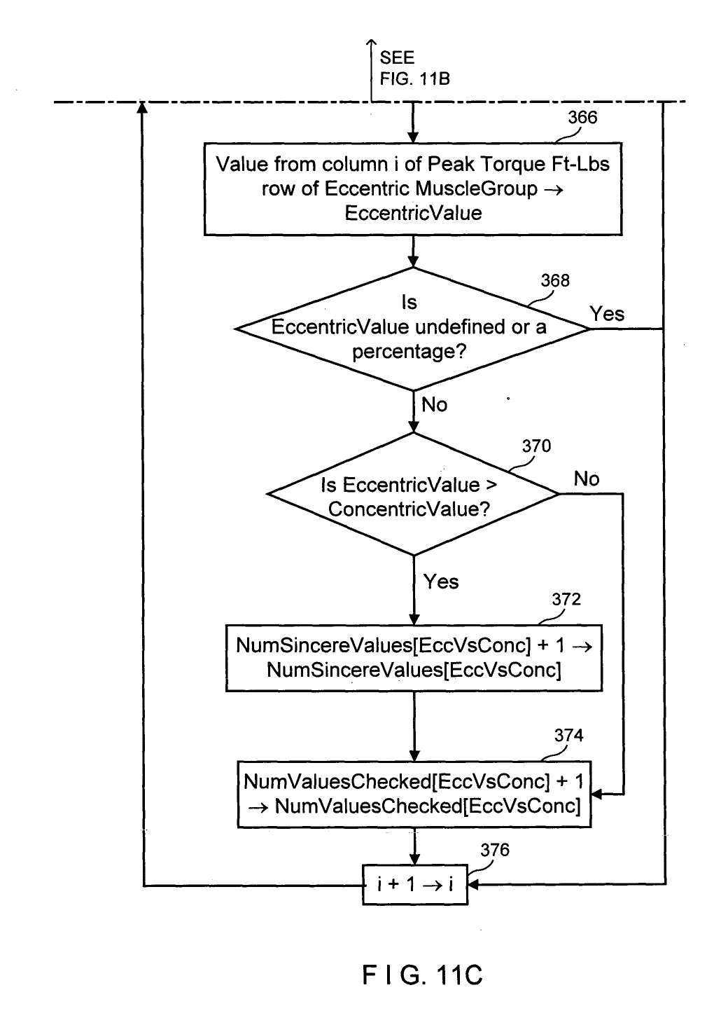

Next, a search is made for the numeric report file which is prepared by the Cybex 6000. It is read by the program at READ THE CYBEX NUMERIC REPORT FILE block 68 and the information is extracted from it which enables the remaining four factors to be calculated. The numerators and denominators of these four factors are then calculated by the four functions CHECK ASD ROWS block 70 (See FIG. 8), CHECK TORQUE VS SPEED block 72 (see FIG. 9), CHECK POWER VS SPEED block 74 (see FIG. 10) and CHECK ECCENTRIC VS CONCENTRIC block 76 (see FIG. 11).

This completes the evaluation of the numerators and denominators of all seven of the factors, and each factor is now available for calculating the final index. The loop which follows blocks 78 through 86 is executed once for each factor and, in the present example, seven times.

The first item in the loop is to check at IS NUM VALUES CHECKED block 78 entry for the factor is still zero. This indicates that no values were found from which the factor could be calculated. That factor is then dropped from the calculation. If the numerical values checked is not zero, then the sincerity factor is calculated at NUM SINCERE VALUES/NUM VALUES CHECKED [i]→SINCERITY [i] block 80. This factor is multiplied by the weight at SINCERITY [i]×WEIGHT [i]+FINAL SCORE→FINAL SCORE block 82 and this factor is then contributed to the final score. These factor contributions are accumulated by this process to obtain the final score for the subject. Also, the weight values are summed at the WEIGHT [i]→WEIGHT SUM−WEIGHT SUM block 84 for those factors which had non-zero denominators to give weight sum—a number between 0 and 7. The final sincerity score for the subject is then completed at FINAL SCORE/WEIGHT SUM→FINAL SCORE block 92 and at EXIT block 94.

Timing Factor

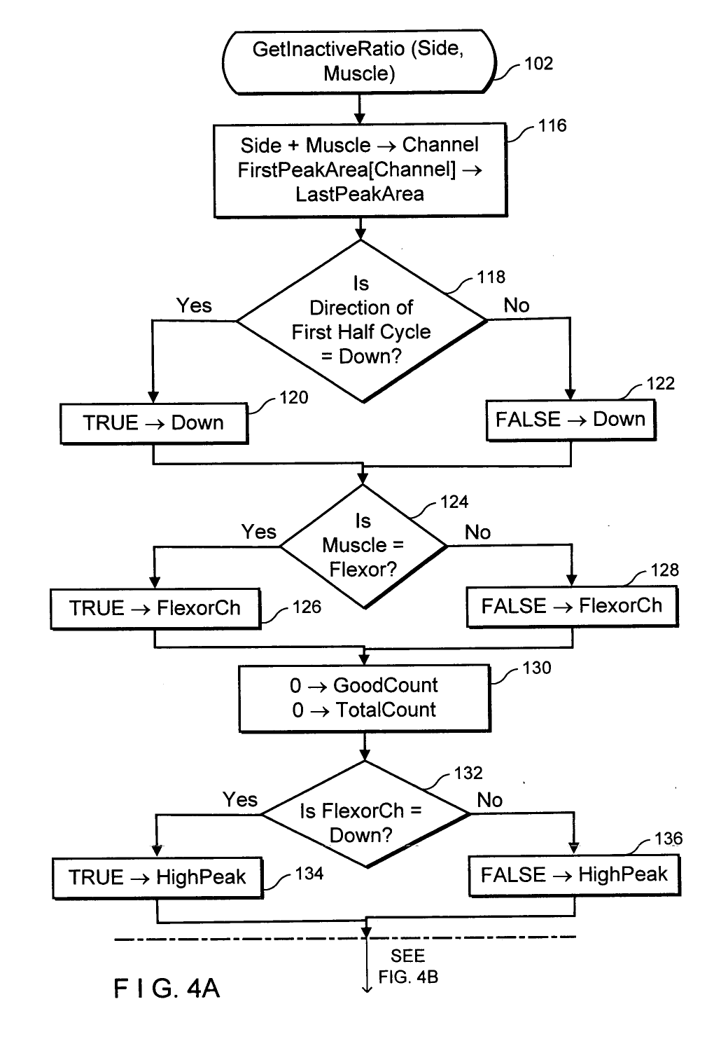

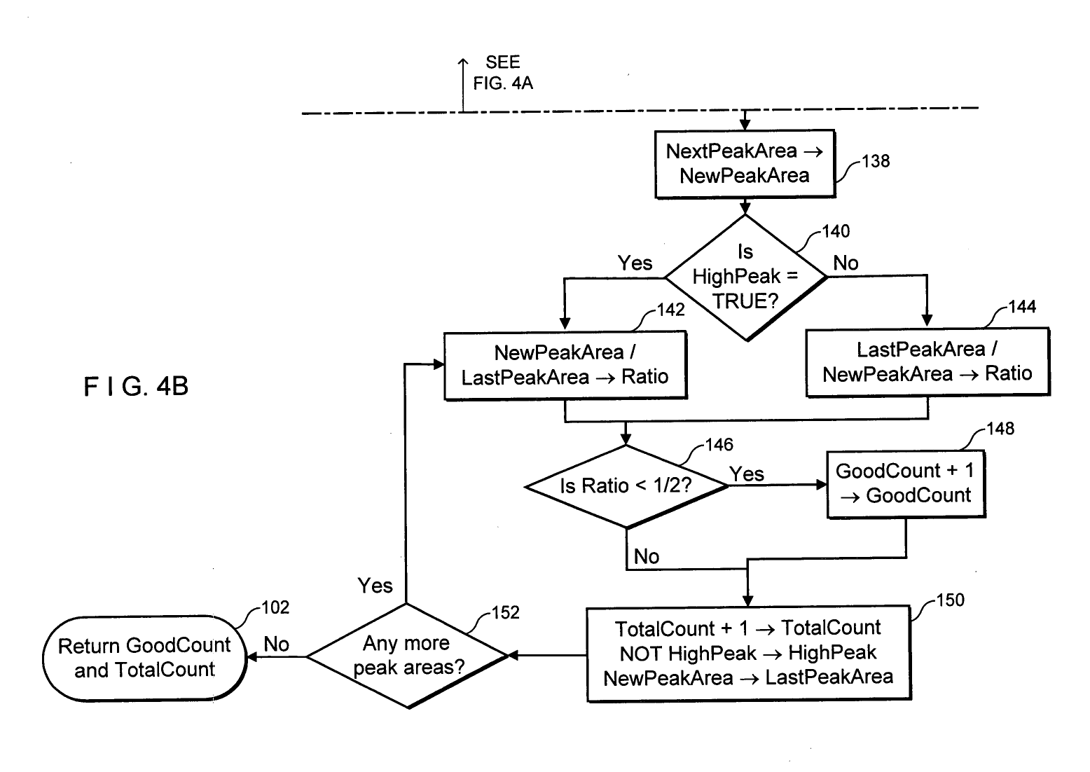

Referring now to FIGS. 3 and 4 a flow chart is shown providing the details of the CALCULATE TIMING FACTOR block 60 of FIG. 2 and the subprogram thereof, the GETINACTIVE RATIO block 102 of FIG. 3. This function is an element of the sincerity analysis of this invention. The timing factor is derived from the sEMG data, and, when an individual is sincere in performing the test activity, the opposing muscles are not contracted at the same time. This test compares the area under the graph for two opposing muscles for each direction of the action. It then verifies that the appropriate muscle has the larger area under the graph.

$$Timing Factor = \frac{U + D}{U + D} ,$$

where

U=Number of times extensor peak area≦flexor peak area during up actions,

D=Number of times extensor peak area<flexor peak area during down actions,

U=Total number of up actions and

D=Total number of down actions.

The only actions which contribute to the timing factor are those actions which are performed at speeds below 70 degrees per second. Thus, at IS SPEED<70 DEG/SEC? Block 96, this threshold level is ascertained and, as faster actions are ignored by this function, when higher speeds are encountered, the loop ends at EXIT block 94. If the speed is slow enough, contributions to the timing factor are calculated for four muscles. The contribution for each muscle is calculated by the function GET INACTIVE RATIO block 102 which is called four times. First GET INACTIVE RATIO is called for the left side extensor muscle, by LEFT→SIDE block 98 and EXTENSOR→MUSCLE block 100, respectively and thereafter for the left flexor muscle, then for the right extensor muscle, and finally for the right flexor muscle. The sequential stepping through of these muscles occurs by the logical progression through the IS MUSCLE? EXTENSOR? Block 106, the FLEXOR→MUSCLE block 108, IS SIDE=LEFT? Block 110 and RIGHT→SIDE block 112.

Each time that GET INACTIVE RATIO block 102 is called, it returns a value of GOOD COUNT block 102 for the number of tests that were sincere, and TOTAL COUNT for the number of tests that were performed. The values GOOD COUNT and TOTAL COUNT from block 102 are added to the appropriate entry in the arrays NUM SINCERE VALUES and NUM VALUES CHECKED block 104.

Get Inactive Ratio

GET INACTIVE RATIO block 102 first finds the CHANNEL block 116 in the graphical file for the side and muscle requested. The are of the first peak for that channel is assigned to the variable LAST PEAK AREA block 116. If the direction of motion while the first peak was being generated is down, as determined IS DIRECTION OF FIRST HALF CYCLE=DOWN? BLOCK 118, then the variable DOWN is given the value of TRUE at TRUE→DOWN block 120. Otherwise, at FALSE→DOWN block 122 the value is set to FALSE.

If the requested muscle is a flexor muscle, as determined at IS MUSCLE=FLEXOR? Block 124, then the variable FLEXOR CH is set to TRUE at TRUE→FLEXOR CH block 126. otherwise it is set to FALSE at FALSE→FLEXOR CH block 128.

The return values GOOD COUNT and TOTAL COUNT are then initialized to zero at 0→GOOD COUNT, 0→TOTAL COUNT block 130.

As ascertained at IS FLEXOR CH=DOWN? Block 132, if the variables DOWN and FLEXOR CH both have the same value, then at TRUE→HIGH PEAK block 134 the variable HIGH PEAK is set to TRUE; otherwise, at FALSE→HIGH PEAK block 136 it is set to FALSE. That is to say, HIGH PEAK is set true for down motions of flexor muscles or up motions of extensor muscles. It is set false for down motions of extensor muscles or up motion of flexor muscles.

The function then enters a loop at NEXT PEAK AREA→NEW PEAK AREA block 138 in which the areas of adjacent peaks are compared. The loop starts by assigning the area of the next peak to the variable NEW PEAK AREA. Then, if at IS HIGH PEAK=TRUE block 140 the ratio is calculated at NEW PEAK AREA/LAST PEAK AREA→RATIO block 142. If HIGH PEAK is false, the ratio is calculated at LAST PEAK AREA/NEW PEAK AREA→RATIO block 144. If the ratio is less than 0.5 at IS RATIO<½? Block 146, then a sincere pair of peaks has been found and GOOD COUNT is incremented by one at GOOD COUNT+1→GOOD COUNT block 148. In either case, at block 150, TOTAL COUNT is incremented by one. The value of HIGH PEAK is then inverted (FALSE goes to TRUE, and TRUE goes to FALSE) and NEW PEAK AREA is saved as LAST PEAK AREA. The loop at ANY MORE PEAK AREAS block 152 is then repeated until there are no more peaks for the channel and returns to GOOD COUNT, TOTAL COUNT block 102.

The comparison of the areas of adjacent peaks is significant because, when the HIGH PEAK is TRUE, the corresponding peak should be over twice the area of the following peak for a sincere individual; and conversely, when FALSE, the peak should be less than half the area of the following peak.

Fatigue Factor

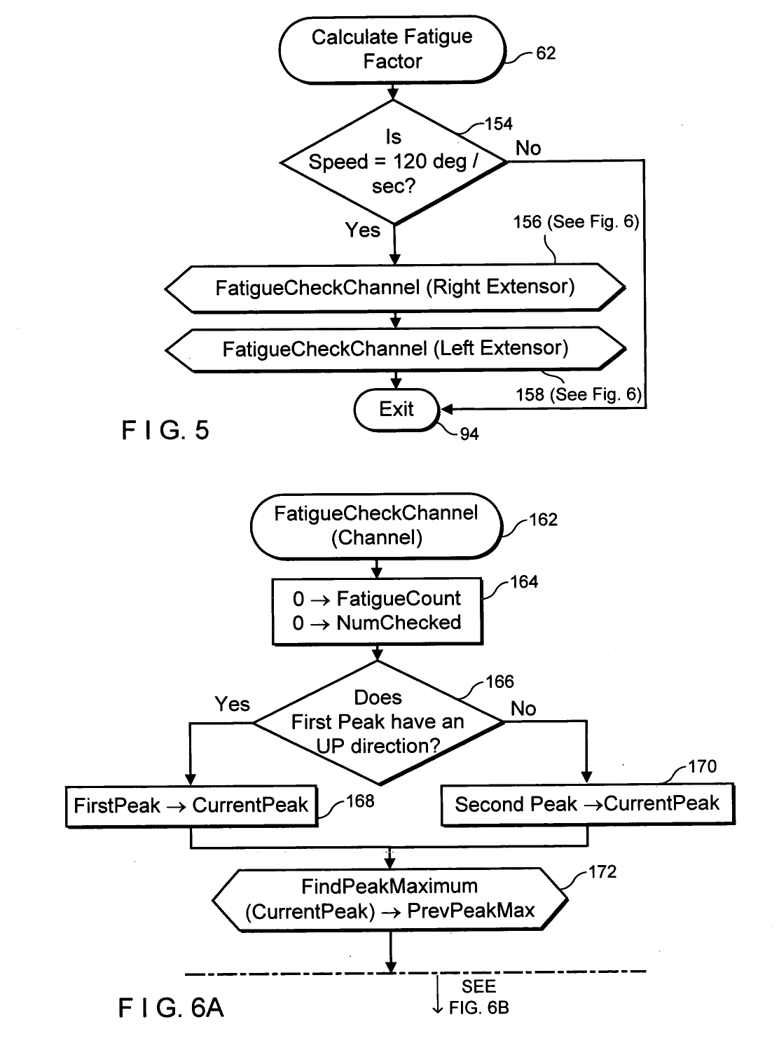

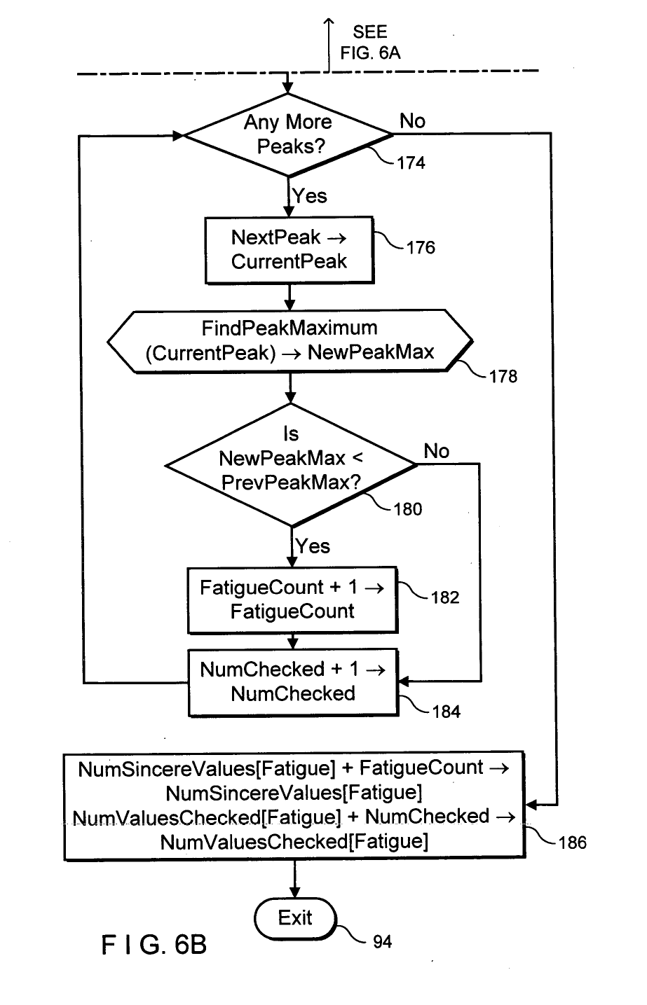

Referring now to FIGS. 5 and 6 a flow chart is shown providing the details of the CALCULATE FATIGUE FACTOR block 62 of FIG. 2 and the subprogram thereof, the FATIGUE CHECK CHANNEL block 156 of FIG. 5. This function is an element of the sincerity analysis of this invention. One of the physical activity tests is performed 20 or so times at quite a high rate of speed. The test lasts for about 40 seconds. This is enough work, done over enough time for a fatigue effect to be discernible. The successive peaks should gradually decrease in amplitude over the course of the test if a maximum effort is being made. When a maximum effort is not being made, the successive peak heights can vary quite widely. This test is only made on the extensor muscles.

Fatigue Factor=Number of times that P<P for 1≦i≦n−1, where

n the number of peaks and

P the maximum height of peak I

The fatigue factor is only calculated for files in which the speed exceeds the threshold of 120 degrees per second, which threshold is determined at IS SPEED=120 DEG/SEC block 154. Other files make no contribution to the fatigue factor. The bulk of the fatigue factor calculation is done by the FATIGUE CHECK CHANNEL function which is called twice—once for the right extensor muscle at block 156 and once for the left extensor muscle at block 158. If the threshold is not met and upon completion of CALCULATE FATIGUE FACTOR block 62, the program exits at EXIT block 94.

Fatigue Check Channel

The FATIGUE CHECK CHANNEL function 162, FIG. 6, starts by setting the variables to zero at 0→FATIGUE COUNT, 0→NUM CHECKED block 164. If the first peak on the channel for the requested muscle has an up direction then the variable current peak is set to the first peak at FIRST PEAK→CURRENT PEAK block 168. Otherwise the variable is set to the second peak at SECOND PEAK→CURRENT PEAK block 170. Then, the variable PREV PEAK MAX is set to the maximum value of the peak indicated by CURRENT PEAK at FIND PEAK MAXIMUM (CURRENT PEAK)→PREV PEAK MAX block 172.

The function then enters a loop beginning at ANY MORE PEAKS? block 194 in which the maximum values of adjacent, up direction peaks are compared. The loop starts by changing CURRENT PEAK to point to the next up direction peak at NEXT PEAK→CURRENT PEAK block 176, and then finds the maximum value of CURRENT PEAK, saving the result as NEW PEAK MAX block 178.

If NEW PEAK MAX is less than PREV PEAK MAX at IS NEW PEAK MAX <PREV PEAK MAX block 180, then a sincere pair of peaks has been found. Upon such an occurrence, FATIGUE COUNT is incremented by one at FATIGUE COUNT+1→FATIGUE COUNT block 182. In any event, NUM CHECKED is incremented by one at NUM CHECKED+1→NUM CHECKED block 184. Provided that the channel still has two unchecked peaks left the loop through ANY MORE PEAKS? block 174 is then repeated.

After the whole channel has been checked, FATIGUE COUNT is used to increment the appropriate entry in NUM SINCERE VALUES and NUM CHECKED is used to increment the corresponding entry of NUM VALUES CHECKED at block 186. Upon entering the fatigue factor, the program exits at EXIT block 94. This factor monitors the gradual decrease as the subject tires for a sincere individual of the maximum values of up direction peaks.

Force Factor

Referring now to FIG. 7 a flow chart is shown providing the details of the CALCULATE FORCE SINCERITY block 66 of FIG. 2. This function is an element of the sincerity analysis of this invention. The tests are performed at three of four different speeds. For a sincere individual, the force exerted should be decreased as the speed increases. This test is done by averaging the areas under the peaks of the extensor graphs for each repetition in order to get an average peak area for each speed.

$$Force Factor = \frac{Number of times that A > A}{n - 1} ,$$

for 1≦I≦n−1, where

Ai=the average peak area for speed I and

n=the number of speeds

The force factor depends on a comparison of the average peak areas of activities performed at different speeds. Consequently it cannot be done until all the graphical files have been read by the program. As the file for each activity is read, a record is made of the speed of the activity in degrees per second and the average peak area of the graphs.

It is possible that some activity speeds were performed more than once by the subject. Thus, while the following is exemplary, the first phase in calculating the force sincerity factor is to find the average peak area for each speed by combining the averages from the individual files. This is done by reviewing the records for the individual files and summing the average peak areas for each speed. Then the sums are divided by the number of files at that speed.

An array is kept, called AVERAGE PEAK AREA SUM which has an entry for each possible speed. There is a corresponding array called NUM PEAK AVERAGES which is used to keep track of the number of average peak areas which have been summed for any given speed.

Calculate Force Sincerity

CALCULATE FORCE SINCERITY block 66 is the function which carries out the main work of calculating the force factor. First, the arrays AVERAGE PEAK AREA SUM and NUM PEAK AVERAGES are cleared to zero at block 190. Next, a loop is entered at START WITH THE FIRST FILE READ block 192 which runs through the records for each file. The files are then sorted at WAS FILE FOR ONE OF THE REPETITIVE ACTIVITIES block 194, and files which are not for one of the repetitive activities are bypass this loop. For the repetitive activity files at block 196, the activity speed is extracted to serve as an index to the two arrays. Then the average peak area for the file is extracted, and added to the appropriate entry of AVERAGE PEAK AREA SUM. Next, the corresponding entry of NUM PEAK AVERAGES is incremented by one. This procedure is then repeated for each file which had been reviewed earlier by stopping through ANY MORE FILES? Block 198 and STEP ON TO NEXT FILE block 200.

When all the file records have been processed, a second loop starting at 0→SPEED block 202 is entered, which loop is executed once for each possible speed. For each speed, an entry in the AVERAGE PEAK AREA SUM array is divided by the corresponding entry in the NUM PEAK AVERAGES array to give a final average peak area at block 204. These are saved in the AVERAGE PEAK AREA array. Each time through the loop, recycling through the loop is determined by SPEED+1→SPEED block 206 and LAST SPEED? Block 208. When the last speed has been processed, the program is then ready to evaluate the contributions to the force factor.

This is calculated by a third loop that starts at 1→SPEED block 210 The program then reviews the AVERAGE PEAK AREA array, comparing at block 212 the average peak areas for adjacent speed entries. Each time through the loop, the average peak area for one speed is compared to the average peak area for the next higher speed. If the average peak area for the slower speed is greater than the average peak area for the next higher speed, then the test is considered sincere and one is added to the NUM SINCERE VALUES array at block 214 entry for the force factor. In either case, one is added to the NUM VALUES CHECKED array at block 216 entry for the force factor.

In this loop, recycling through the loop is determined by SPEED+1→SPEED block 218 and LAST SPEED? block 220. Upon entering the force factor, the program exits at EXIT block 94. This factor is significant as a sincere individual exerts more force at slower speeds which, in turn, provides average peak areas in proportion to the force exerted.

Average Standard Deviation Factor

Referring now to FIG. 8 a flow chart is shown providing the details of the average standard deviation (ASD) factor, the CHECK ASD ROWS block 70 of FIG. 2. This function is an element of the sincerity analysis of this invention. Each test is performed with a number of repetitions. The average standard deviation of the torque between repetitions is calculated for each physical activity. This value is proportional to the amount of variation between the repetitions. An average standard deviation greater than seven indicates an unreasonably high amount of fluctuation. The probable cause is that the amount of effort put into the physical activity is also varying widely. The average standard deviation sincerity factor is the ratio

$$ASD Factor = \frac{\begin{matrix}Number of average standard deviation \\ values which are < 7\end{matrix}}{\begin{matrix}Total number of average standard deviation \\ values available\end{matrix} }$$

The average standard deviation factor is calculated by examining the average standard deviation values which are contained in the 9-column numerical report which is produced by the Cybex 6000. Although the following discussion of the Average Standard Deviation Factor is based on a particular force dynamometer, the Cybex 6000, the program may be adapted to run on numerical reports from instrumentation by other manufacturers. The calculation is performed by two nested loops. The outer loop, which is controlled by the index I, is executed twice, once for each of the muscle groups shown on the report and is entered at 0→I block 224.

The inner loop is controlled by the index j, is started at 0→j block 226 and is executed nine times, once for each possible column on the report. The working part of the loop is therefore concerned with column j of muscle group I of the report.

The first thing done inside the loop is to extract the value of column j of the average standard deviation (ASD) row of muscle group I at block 228. If this value is undefined or a percentage value (see block 230) then it is discarded and the loop steps on to the next column. If the value is defined, and is not a percentage, it is tested at IS VALUE<7? Block 232 to see if it is less than seven. If so, the value is considered sincere and one is added to the NUM SINCERE VALUES block 234 array entry of the ASD factor. In either case, one is added to the NUM VALUES CHECKED block 236 array entry for the ASD factor.

The inner loop then by stepping through columns at j+1→j block 238 and ANY MORE COLUMNS? Block 240 carries on checking the remaining values in the ASD row. Then the outer loop ascertains the ASD row of the second muscle group by stepping from one to the other at I+1→I block 242 and ANY MORE MUSCLE GROUPS? Block 244. Upon calculation and the entry of the ASD factor, the program exits at EXIT block 94. Here the significance arises as sincere individuals are less erratic than insincere individuals. Therefore sincere individuals have smaller average standard deviations. Seven is the breakover point from sincere to insincere.

Torque Vs Speed Factor

Referring now to FIG. 9 a flow chart is shown providing the details of the CHECK TORQUE vs. SPEED block 72 of FIG. 2. This function is an element of the sincerity analysis of this invention. The maximum torque achieved by the individual over all repetitions of some action at a given speed should decrease as the speed increases. A low value of peak torque occurring at a slow speed is indicative of little effort being made by the individual. The torque vs. speed factor is the ratio

$$Torque vs Speed Factor = \frac{\begin{matrix}Number of times that peak torque \\ decreases with speed\end{matrix}}{Number of speeds - 1}$$

In a manner similar to the ASD Factor, the TORQUE VS SPEED factor is also calculated from the values contained on the numeric report from the Cybex 6000 or equivalent dynamometer. The calculation is done by three nested loops. The outer loop is controlled by the index I, and is executed twice, once for each of the two muscle groups on the report and is started at 0→I block 248.

The middle loop is controlled by the index j and is started at 0→j block 250. It searches at blocks 252 and 254 across the columns of muscle group I looking for a column j which has a value in both the speed row and the peak torque ft-lbs row which is not a percentage and is defined.

When such a column j has been identified, the speed and peak torque ft-lbs values are saved at block 256 as the variables 2ND SPEED and 2ND TORQUE. After at block 258 ascertaining that 2ND TORQUE is not a percentage and is defined the inner loop is started at j+1→k block 260. The inner loop is then entered. This loop is controlled by the index k.

The inner loop then at block 270 saves the values 2ND SPEED and 2ND TORQUE as the variables 1ST SPEED and 1ST TORQUE. Next then after determining at block 272 that 2ND SPEED is not a percentage and is defined, the values from the speed row and the peak torque ft-lbs row of column k and muscle group I are extracted and saved as 2ND TORQUE at block 274. If they are both values which are defined and are not percentages, as determined at block 276 then they are compared against the. values 1ST SPEED and 1ST TORQUE at blocks 278 and 280.

If 1ST SPEED<2ND SPEED and 1ST TORQUE>2ND TORQUE or 1ST SPEED>2ND SPEED and 1ST TORQUE<2ND TORQUE then a sincere test is indicated, and one is added to the TORQUE VS SPEED entry of the NUM SINCERE VALUES array at block 282. In either case, one is added to the TORQUE VS SPEED entry of the NUM VALUES CHECKED array at block 284. This factor is significant because, for a sincere individual, the peak torque value decreases as the speed increases, and conversely, the peak torque value increases as the speed decreases.

If k at block 286 is not the last column in the report then k is increased by one at k+1→k block 288 and the inner loop is repeated.

If k is the last column in the report, or a column k is found which does not contain defined values which are not percentages in both the speed row and the peak torque ft-lbs row then the inner loop is exited after j is set to the value of k at block 292.

The middle loop then increments j by one at j+1→j block 262 and continues as before, looking for suitable values of speed and peak torque ft-lbs, blocks 252 through 260, with which to do more sincerity comparisons. When j reaches the value of 8 or more, the middle loop is exited and the outer loop is reentered. This increments I by 1, and, provided I has not reached a value of w, the middle loop is restarted to check the next muscle group on the report. If I has reached 2 the check TORQUE VS SPEED function is completed and the program exits at EXIT block 94.

Power Vs Speed Factor

Referring now to FIG. 10 a flow chart is shown providing the details of the CHECK POWER vs. SPEED block 74 of FIG. 2. This function is an element of the sincerity analysis of this invention. The average power in the best work repetition is calculated at each speed. These should increase as the speed increases. Any cases where the average power decreases when the speed has increased are probably caused by lack of effort.

$$Power vs Speed Factor = \frac{\begin{matrix}Number of times that power \\ increases with speed\end{matrix}}{Number of speeds - 1}$$

The calculation of the POWER VS SPEED factor is similar to the calculation of the TORQUE VS SPEED factor. It also is composed of three nested loops. Once again, the outer loop is controlled by index I, and is executed twice, once for each muscle group on the numeric Cybex 6000 or equivalent dynamometer report and is started at 0→1 block 296.

Access to the middle loop is controlled by the index j at 0→j block 302. The program at block 310 scans across the columns of the report from column 0 to column 7, the next to last column. As it scans across the columns, it extracts the values from the speed row. After determining at block 312 that 2ND SPEED is not a percentage and is defined, the average power watts row of column j of muscle group I. These values are saved as 2ND SPEED at block 310 and 2ND POWER at block 314. If 2ND SPEED is undefined or a percentage at block 312 or if 2ND POWER is undefined or a percentage at block 316 are rejected, j is incremented at j+1→j block 308 by one and the next column is examined. If they are both defined values which are not percentages, then the inner loop is entered through j+1→k block 318.

The inner loop is controlled by the index k which as just stated is initialized to the value of j+1 and at block 320 the program checks that there are remaining columns to be scanned. Then the inner loop at block 322 saves the values of 2ND SPEED and 2ND POWER as the variables 1ST SPEED and 1ST POWER. Next, if 2ND SPEED is undefined or a percentage of block 324, the values are extracted from the speed row and the average power watts rows of column k and muscle group I. These values are saved as 2ND SPEED and 2ND POWER at block 326. If 2ND POWER is defined and not a percentage, then they are compared with the values 1ST SPEED and 1ST POWER at blocks 330 and 332.

If 1ST SPEED<2ND SPEED and 1ST POWER<2ND POWER or 1ST SPEED>2ND SPEED and 1ST POWER>2ND POWER then a sincere test is indicated, and one is added to the POWER VS SPEED entry of the NUM SINCERE VALUES array at block 334. In either case, one is added to the POWER VS SPEED entry of the NUM VALUES CHECKED array at block 336. This factor is significant, because for a sincere individual, the average power value increases as the speed increases, and conversely, the average power value decreases as the speed decreases.

If k is not the last column in the report, then k is increased by one at k+1→k block 338 and the inner loop is repeated.

If at block 320 k is the last column in the report, or if at blocks 324 and 328 a column k is found which does not contain defined values which are not percentages in both the speed row and the average power watts row then the inner loop is exited after at k→j block 340, j is set to the value of k.

The middle loop then at j+1→j block 308 increments j by one, and continues as before, looking at blocks 310 through 316 for suitable values of speed and average power watts with which to do more sincerity comparisons. When j reaches the value of 8 or more, the middle loop is exited at block 302 and the outer loop is reentered. This at I+1→I block 304 increments I by 1, and, provided I has not reached a value of 2, the middle loop is restarted to check the next muscle group on the report. If I has reached 2, the CHECK POWER VS SPEED function is completed and the program exits at EXIT block 94.

Eccentric Vs Concentric Peak Torque Factor

Referring now to FIG. 11 a flow chart is shown providing the details of the ECCENTRIC vs. CONCENTRIC block 76 of FIG. 2. This function is an element of the sincerity analysis of this invention. The peak torque when performing an eccentric activity should be greater than the peak torque when the same muscle group is performing a concentric activity at the same speed. Cases in which this is not observed results from lack of sincerity.

$$\begin{matrix}Eccentric \\ vs . \\ Concentric \\ Factor\end{matrix} = \frac{\begin{matrix}Number of speeds where eccentric peak torque > \\ concentric peak torque\end{matrix}}{Number of speeds tested}$$

The eccentric vs. concentric factor is a comparison between the two muscle groups 0 and 1 on the numeric report produced by the Cybex 6000 or equivalent dynamometer.

To derive this factor MUSCLE GROUP 0 and MUSCLE GROUP 1 are operative in opposite modes of activity, that is, one is operating in an eccentric mode and the other in the concentric mode. Furthermore, both MUSCLE GROUP 0 and MUSCLE GROUP 1 are of the same type, that is, extensors or flexors.

If at block 342 MUSCLE GROUP 0 is not from an extensor or a flexor muscle group, then the function exits at EXIT block 94. If at block 344 the extensor/flexor type of MUSCLE GROUP 1 is not the same as for MUSCLE GROUP 0, then the function exits at EXIT block 94. If at block 346 MUSCLE GROUP 0 is not eccentric or concentric, then the function exits at EXIT block 94. If at block 348 MUSCLE GROUP 1 is not eccentric or concentric, then the function exits at EXIT block 94. If at block 350 MUSCLE GROUP 0 is eccentric and MUSCLE GROUP 1 is eccentric, then the function exits at EXIT block 94. If at block 350 MUSCLE GROUP 0 is concentric and MUSCLE GROUP 1 is concentric, then the program exits at EXIT block 94.

If these conditions are met, then the eccentric vs concentric factor is calculated. First the program determines which muscle group is concentric and which muscle group is eccentric. If at block 352 MUSCLE GROUP 0 is concentric, then variable 0→CONCENTIC MUSCLE GROUP block 354 is set to 0. Otherwise 1→CONCENTIC MUSCLE GROUP block 356 is set to 1. Since the other muscle group must be the eccentric muscle group, the variable ECCENTIC MUSCLE GROUP is set to 1-CONCENTIC MUSCLE GROUP at block 358.

Next, a loop is entered which scans across the columns of the report. The entry to the loop is controlled at block 364 by index I which is initialized to 0. Inside the loop, the values are extracted at blocks 362 and 366 from column I of the peak torque ft-lbs row of both the CONCENTIC MUSCLE GROUP and the ECCENTIC MUSCLE GROUP. They are saved as CONCENTRIC VALUE and ECCENTRIC VALUE. If at blocks 364 and 368 both these values are defined and not percentages, then they are compared. If ECCENTRIC VALUE>CONCENTRIC VALUE block 370 then a sincere test is indicated, and one is added to the ECC VS CONE entry of the NUM SINCERE VALUES array at block 372. In either case, one is added to the ECC VS CONE entry of the NUM VALUES CHECKED array block 374. For a sincere individual, eccentric peak torque values is greater than concentric peak torque values.

Then i at i+1→i block 376 is incremented by one. If i at block 360 is less than the number of columns on the report, the loop is repeated, and the next pair of values is compared.

If either of the pair of values is undefined or a percentage, then the comparison is not performed and I is incremented to the next column immediately. When all the columns have been checked, the ECCENTRIC VS CONCENTRIC function has been calculated and the program exits at EXIT block 94.

Because many varying and different embodiments may be made within the scope of the inventive concept herein taught, and because many modifications may be made in the embodiments herein detailed in accordance with the descriptive requirement of the law, it is to be understood that the details herein are to be interpreted as illustrative and not in a limiting sense.

What is claimed is:

1. A sincerity index system for determining the sincerity of a person undergoing muscular testing, said person having specific first muscles associated with a body movement and second muscles corresponding to said first muscles during the performance of said body movement, said system comprising:
a muscular force detector for sensing and measuring the force exerted by said first muscles upon a predetermined body movement, said force detector having a first signal output proportional to the force exerted;
an electromyograph for sensing and measuring the bioelectric (EMG) activity of said second muscles, said electromyograph, simultaneously with said force detector, having a second signal output proportional to the EMG; and,
an interface device for processing said first and said second signal outputs, said interface device providing at least two factors; and,
an index-forming program for combining said factors in a predetermined manner to provide a sincerity index having a lower limit and an upper limit.

2. A sincerity index system as described in claim 1 wherein said index-forming program further comprises:
a dynamometer force factor program deriving at least one first factor from said first signal, said first factor having a value between said lower limit and said upper limit;
an EMG force factor program deriving at least one second factor from said second signal having a value between said lower limit and said upper limit;
whereby the index formed by the combination thereof represents the sincerity of effort.

3. A sincerity index system as described in claim 2 for determining the sincerity of a person undergoing muscular testing as described in claim 1 wherein said correlative value is an index between 0 and an upper limit with values approaching 0 representing lack of sincerity and those values approaching said upper limit representing greater sincerity.

4. A system for determining the sincerity of a person undergoing muscular testing as described in claim 2 wherein said index further comprises:
a muscular force factor being less than said upper limit with a first fractional weighting factor therfor; and,
an sEMG factor being less than said upper limit with a second fractional weighting factor therefor, said first and said second weighting factors totalling said upper limit.

5. A system for determining the sincerity of a person undergoing muscular testing as described in claim 1 wherein said comparator further comprises:
first program means for analyzing said first signal output to generate said muscular force factor for in-process calculation of the index; and,
second program means for analyzing said second signal output to generate said sEMG factor for in-process calculation of the index.
